(19) United States
(12) Patent Application Publication
(10) Pub. No.: US 2025/0059116 A1
(43) Pub. Date: Feb. 20, 2025

(54) PROCESS FOR PRODUCING PROPOFOL

(71) Applicant: PCAS, ECULLY CEDEX (FR)

(72) Inventors: Jean-Marie SCHNEIDER, MAGNANVILLE (FR); Sandrine GAUTHIER, BREUILPONT (FR); Alexandre DUPAS, MEUDON LA FORET (FR)

(73) Assignee: PCAS, ECULLY CEDEX (FR)

(21) Appl. No.: 18/720,787

(22) PCT Filed: Dec. 16, 2022
(86) PCT No.: PCT/FR2022/052405

§ 371 Date: Jun. 17, 2024

(30) Foreign Application Priority Data
Dec. 17, 2021 (FR) FR2113836

(51) Int. Cl. C07C 29/147

(52) U.S. Cl. CPC C07C 29/147

(57) ABSTRACT

The invention relates to a process for producing 2,6-diisopropyl phenol. The process comprises a step of decarboxylation of 4-hydroxy-3,5-diisopropylbenzoic acid or a salt thereof in an aqueous medium under pressure.

### FIELD OF THE INVENTION

The invention concerns a process for preparing propofol.

### STATE OF THE ART

Propofol is a general anaesthetic with rapid action and short duration of action. It was developed in the 1970s by Glenn. It is administered intravenously.

Consequently, for its pharmaceutical use, its degree of purity must be very high (typically >99.7%). Numerous methods for preparing and purifying this compound have been proposed. For example, it can be prepared from phenol and propene by Friedel-Crafts alkylation. However, the yields are not high and the operating conditions involve high pressures and high temperatures. Moreover, this synthetic route leads to numerous impurities, due especially to the reactivity of phenol not only in ortho positions of the hydroxyl, but also in para position of the hydroxyl and on the hydroxyl itself. These impurities have similar boiling points and are therefore difficult to remove.

To overcome this lack of selectivity during alkylation, it was proposed to prepare propofol by alkylation of a phenol substituted by a para group followed by removal of the para group from the phenol. The starting substrate can be 4-chlorophenol or 4-hydroxybenzoic acid. In particular, in the second case, in a first step, 4-hydroxybenzoic acid is alkylated in positions 3 and 5 to obtain 4-hydroxy-3,5-diisopropylbenzoic acid, which is then decarboxylated in a second step. This synthetic route is especially described in patent SU443019, in patent applications WO 2011/161687, WO 2013/035103, IN1420/MUM/2012, CN106588576 and WO 2021/191832 or, alternatively, in the article by Pramanik et al., “Commercial Manufacturing of Propofol: Simplifying the Isolation Process and Control on Related Substances” Org. Process Res. Dev. 2014, 18, 152-156 or the article by Mougeot et al., “Continuous flow synthesis of propofol” Molecules 2021, 26, 7183.

In SU443019, alkylation takes place in the presence of sulfuric acid, water and isopropyl alcohol. 4-hydroxy-3,5-diisopropylbenzoic acid is isolated by precipitation in the reaction medium and rinsing with water. Decarboxylation takes place in triethylamine at 120-140° C. for 1 hour. According to this process, numerous impurities are present and difficult to remove.

In WO 2011/161687, the number of impurities is reduced by purifying the intermediate product, 4-hydroxy-3,5-diisopropylbenzoic acid, by acid-base washing, washing with water or with a water/methanol mixture and/or recrystallization in a water/methanol mixture. The second decarboxylation step takes place in the presence of alkali metal hydroxide in a high boiling point solvent at high temperature (140-145° C.). The high boiling point solvent can be ethylene glycol, dimethylformamide or dimethylacetamide.

In WO 2013/035103, Friedel-Crafts alkylation takes place in the presence of an acid followed by acid-base washing, precipitation in acid medium, washing with water and recrystallization in a methanol/water mixture to obtain 4-hydroxy-3,5-diisopropylbenzoic acid. Decarboxylation takes place in the presence of a catalyst in a high boiling point organic solvent at high temperature (140-145° C.).

In IN1420/NUM/2012 and the article by Pramanik et al., 4-hydroxybenzoic acid is alkylated in the presence of acid. 4-hydroxy-3,5-diisopropylbenzoic acid is extracted in toluene, precipitated in an alcohol/water mixture and rinsed with a non-polar solvent. The decarboxylation step takes place in 2-ethoxyethanol in the presence of alkali metal hydroxide at a temperature comprised between 12° and 160° C.

In CN106588576, Friedel-Crafts alkylation takes place in the presence of a solid acid under ultrasound and without solvent. 4-hydroxy-3,5-diisopropylbenzoic acid is obtained by filtration and precipitation. The decarboxylation step is catalysed by an enzyme in a buffered aqueous medium for 1 to 6 days.

In WO2021/191832, 4-hydroxybenzoic acid is alkylated in the presence of acid according to the article by Pramanik et al. to obtain 4-hydroxy-3,5-diisopropylbenzoic acid. This acid is carboxylated in the presence of a heterocyclic base, in particular imidazole and optionally in the presence of solvent. This process also leads to the production of numerous organic wastes. In the Article by Mougeot et al., the alkylation step and the decarboxylation step each take place in a continuous flow. Friedel-Crafts alkylation is carried out in the presence of acid and isopropanol. Decarboxylation takes place in the presence of an organic base in various organic solvents, in particular 2-butoxyethanol.

The processes described require multiple stages of isolation and purification of the intermediate and product, long reaction times or the use of flammable solvents.

Thus, a need remains for an efficient, simple, more environmentally friendly and economically competitive process to prepare propofol on an industrial scale.

### SUMMARY

The invention relates to a process for preparing 2,6-diisopropyl phenol comprising a step of decarboxylation of 4-hydroxy-3,5-diisopropylbenzoic acid or a salt thereof in an aqueous medium under pressure.

Other aspects of the invention are as described below and in the claims.

### DETAILED DESCRIPTION

The inventors have developed a process meeting the needs expressed. The process for preparing 2,6-diisopropylphenol or 2,6-bis(propan-2-yl)phenol, commonly referred to as propofol, proposed by the inventors comprises a step of decarboxylation of 4-hydroxy-3,5-diisopropylbenzoic acid (also referred to as 4-hydroxy-3,5-di(propan-2-yl)benzoic acid) or a salt thereof in an aqueous medium under pressure.

Propofol is manufactured while limiting the use of toxic solvents (such as 2-ethoxyethanol, ethylene glycol, dimethylformamide or dimethylacetamide) or the use of toxic organic bases (such as triethylamine or imidazole) and while reducing the emission of organic and/or toxic waste.

According to the proposed process, it is also no longer necessary to evaporate toxic organic solvents with a high boiling point generally used in the known processes of the prior art during the decarboxylation step.

In addition, the isolation and purification of propofol is simplified. In fact, at the end of the decarboxylation stage, the propofol is separated directly by decantation from the aqueous reaction medium and then distillation. Extraction is not necessary.

The reaction time of the decarboxylation step is reduced, in particular when the reaction takes place in continuous flow.

Moreover, in continuous flow, propofol is obtained with a degree of purity ranging up to 98% even before purification. The degrees of purity presented here are determined by high performance liquid chromatography (HPLC) according to a method of the European Pharmacopoeia.

### Decarboxylation of 4-Hydroxy-3,5-Diisopropylbenzoic Acid or a Salt Thereof

The decarboxylation step of 4-hydroxy-3,5-diisopropylbenzoic acid or a salt thereof takes place in an aqueous medium under pressure.

The term “under pressure” designates a pressure greater than normal atmospheric pressure, i.e., greater than 0.1 MPa. The pressure can be greater than 0.1 MPa and range up to 3 MPa or range from 0.2 MPa to 2.5 MPa.

The 4-hydroxy-3,5-diisopropylbenzoic acid useful in the process of the present invention can be in the form of salts, such as a mono-salt or a di-salt. Examples of salts include, but are not limited to, alkali metal salts (e.g., lithium, sodium, potassium and caesium salts), alkaline earth metal salts (e.g., calcium, barium, strontium and magnesium salts), or ammonium salts derived from ammonia or from a primary, secondary or tertiary organic amine having from 1 to 20 carbon atoms such as ethylamine, diethylamine, triethylamine, ethyldiisopropylamine or piperidine. The salt of 4-hydroxy-3,5-diisopropylbenzoic acid is preferably a mono- or di-salt of alkali metals. Thus, in certain embodiments, 4-hydroxy-3,5-diisopropylbenzoic acid or a salt thereof useful in the process of the present invention is 4-hydroxy-3,5-diisopropylbenzoic acid or a mono- or di-salt of alkali metals.

The aqueous medium typically consists exclusively of water. However, small amounts of organic solvent may be present in the aqueous medium. The aqueous medium is then said to consist essentially of water. Thus, in certain embodiments, the aqueous medium may comprise, in addition to water, up to 10% by volume of organic solvent, in particular up to 5%, or up to 3%, or up to 2%, or up to 1%, or up to 0.5%, by volume, of organic solvent.

The reaction can take place in the presence of a base, preferably mineral. The use of a base is typically not necessary when the reaction is carried out from a salt of 4-hydroxy-3,5-diisopropylbenzoic acid.

In particular, the base can be a tripotassium hydroxide, carbonate, alkoxide or phosphate. It can be a hydroxide, carbonate or alkoxide of alkali metals or alkaline earth metals. Preferably, the base is an alkali metal hydroxide, an alkali metal carbonate, an alkali metal alkoxide or tripotassium phosphate (K₃PO₄). More preferably, the base is an alkali metal hydroxide.

More particularly, the base is LiOH, NaOH, KOH, tBuOK or K₃PO₄, preferably sodium hydroxide.

The quantity of base typically varies from 0.1 to 3 molar equivalents relative to the quantity of 4-hydroxy-3,5-diisopropylbenzoic acid or a salt thereof, preferably from 0.5 to 2.5 equivalents, more preferably from 1.0 to 2.5 equivalents.

Typically, the decarboxylation step is carried out in the absence of enzyme.

In particular, the decarboxylation step is carried out in less than 18 hours, more particularly between 5 minutes and 18 hours.

The decarboxylation step can take place in batches or in continuous flow.

It can thus be implemented in an autoclave.

When the decarboxylation step takes place in batches, the pressure of the reaction medium of the decarboxylation step preferably varies from 0.2 MPa to 1.2 MPa, preferably from 0.25 MPa to 1.0 MPa. Thus, the decarboxylation step is preferably carried out at a pressure varying from 0.2 MPa to 1.2 MPa, preferably from 0.25 MPa to 1.0 MPa.

When the decarboxylation step takes place in batches, in particular, the decarboxylation step takes place at a temperature ranging from 120° C. to 200° C., preferably from 125° C. to 190° C., more preferentially from 135° C. to 180° C.

When the decarboxylation step takes place in batches, in particular, the decarboxylation step takes place for a period ranging from 30 minutes to 18 hours, preferably from 1 hour to 15 hours.

When the decarboxylation step takes place in continuous flow, the pressure of the reaction medium of the decarboxylation step typically varies from 1.0 MPa to 2.5 MPa, preferably from 1.5 MPa to 2.0 MPa. Thus, the decarboxylation step is preferably carried out at a pressure ranging from 1.0 MPa to 2.5 MPa, preferably from 1.5 MPa to 2.0 MPa.

When the decarboxylation step takes place continuous flow, the decarboxylation step generally takes place at a temperature ranging from 160° C. to 220° C., preferably from 170° C. to 210° C., more preferentially from 190° C. to 210° C.

In particular, when the decarboxylation step takes place in continuous flow, the temperature and pressure are adapted so that the reaction medium is always in the liquid phase.

Typically, in continuous flow, an aqueous solution of 4-hydroxy-3,5-diisopropylbenzoic acid in basic medium is prepared or an aqueous solution of the salt of 4-hydroxy-3,5-diisopropylbenzoic acid is prepared. The resulting aqueous solution is introduced into a continuous reactor. The continuous reactor is, in particular, thermostatically controlled at the required temperature. The aqueous solution is introduced into the continuous reactor at the required pressure. The residence time typically varies from 30 seconds to 20 minutes, preferably from 45 seconds to 15 minutes.

The 4-hydroxy-3,5-diisopropylbenzoic acid useful in the context of the present invention can be prepared according to methods described in the prior art, especially in the article by Pramanik et al. (“Commercial Manufacturing of Propofol: Simplifying the Isolation Process and Control on Related Substances” Org. Process Res. Dev. 2014, 18, 152-156).

In particular, 4-hydroxy-3,5-diisopropylbenzoic acid can be prepared from 4-hydroxybenzoic acid. 4-hydroxybenzoic acid can thus be alkylated in the presence of isopropanol and an acid to give 4-hydroxy-3,5-diisopropylbenzoic acid.

The acid is preferably a mineral acid. It can be selected from the group consisting of hydrochloric acid, perchloric acid and sulfuric acid, in particular, the acid is sulfuric acid.

The molar quantity of acid added generally varies from 8 to 16 equivalents relative to the molar quantity of 4-hydroxybenzoic acid, preferably from 10 to 15 equivalents.

Preferably, the acid is in the form of an aqueous acid solution, in particular, aqueous sulfuric acid. The aqueous sulfuric acid is typically at a concentration ranging from 85% to 98% by weight, preferably from 89% to 98% by weight.

The molar quantity of isopropanol typically varies from 2.0 to 3.5 equivalents relative to the molar quantity of 4-hydroxybenzoic acid, preferably from 2.2 to 3.0 equivalents.

Alkylation is typically carried out at a temperature ranging from 40° C. to 70° C., preferably from 50° C. to 65° C.

The reaction medium during the alkylation step is typically heated for 3 to 24 hours, preferably for 4 to 16 hours.

At the end of the alkylation step, the crude reaction product is treated in order to isolate the 4-hydroxy-3,5-diisopropylbenzoic acid and optionally to purify it.

The treatment can comprise the following steps, notably as described in the article by Pramanik et al. cited above:
a) Addition of an aqueous solution and an organic solvent to the crude reaction obtained from the alkylation step,
b) Separation of the organic phase containing 4-hydroxy-3,5-diisopropylbenzoic acid, c) Optionally, concentration of the organic phase,
d) Optionally, precipitation or recrystallization of 4-hydroxy-3,5-diisopropylbenzoic acid.

The aqueous solution added in step a) can be water.

The organic solvent added in step a) can be toluene, dichloromethane, cyclohexane, heptane or ethyl acetate. Preferably, the organic solvent added in step a) is toluene.

In step d), the precipitation of 4-hydroxy-3,5-diisopropylbenzoic acid can take place by dissolving it in an organic solvent and then adding water. The organic solvent used for the precipitation can be an alcohol or alkane, for example methanol, ethanol, cyclohexane or heptane.

During step d), the recrystallization of 4-hydroxy-3,5-diisopropylbenzoic acid can be carried out in a solvent such as an alkane, in particular heptane.

The resulting 4-hydroxy-3,5-diisopropylbenzoic acid can be directly subjected to the decarboxylation step. Alternatively, the 4-hydroxy-3,5-diisopropylbenzoic acid obtained can be treated beforehand with a basic aqueous phase, in particular an aqueous phase with a pH greater than 9.5. The aqueous phase comprising the salt obtained can be directly subjected to the decarboxylation step or the salt obtained can be isolated.

### Isolation and Purification

At the end of the decarboxylation step, propofol can be isolated and purified by techniques well known to the person skilled in the art. Thus, the process of the present invention can comprise a step of isolating and purifying propofol. Propofol is present in the organic phase formed at the end of the decarboxylation reaction.

The organic phase can thus be separated from the aqueous phase in order to isolate the propofol.

The propofol is advantageously obtained with a degree of purity greater than or equal to 97%, preferably greater than or equal to 98%.

The resulting propofol can then be purified by distillation. The degree of purity obtained after distillation is then 99.9%.

The examples which follow are given by way of illustration. They should in no case be considered as limiting the present invention.

### EXAMPLES

### Example 1: Preparing Propofol by Decarboxylation in Pressurized Water in Batches

4-hydroxy-3,5-diisopropylbenzoic acid (10 g, 45 mmol, 1 equiv) is suspended in water (22 mL) and stirred at room temperature. An aqueous solution of NaOH at 20% weight/volume (2 equiv, 28 mL) is slowly poured in while maintaining the temperature at 20° C., then the reaction medium is held with stirring for 30 min. The reaction medium is transferred into a 100 mL autoclave and then heated for 2 h to the desired temperature indicated in Table 1. The reaction medium is then cooled to room temperature and the organic phase is then decanted. The conversion is then analysed by HPLC and reported in Table 1.

TABLE 1

| | Temperature | Pressure | Conversion |
|---|---|---|---|
| Test | (° C.) | (bar) | (%) |
| 1 | 180 | 9 | 99.7 |
| 2 | 160 | 6 | 99.7 |
| 3 | 140 | 3 | 79 |

### Example 2: Preparing Propofol by Decarboxylation in Pressurized Water in Batches

4-hydroxy-3,5-diisopropylbenzoic acid (10 g, 45 mmol, 1 equiv) is suspended in water (50 mL) and stirred at room temperature. A base (2 equiv.) is added while maintaining the temperature at 20° C., and then the reaction medium is held with stirring for 30 min. The reaction medium is transferred to a 100 mL autoclave and then heated for 2 h at 160° C. The reaction medium is then cooled to ambient temperature and the organic phase is then decanted. The conversion is then analysed by HPLC and reported in Table 2).

TABLE 2

| Test | Base | Conversion (%) |
|---|---|---|
| 1 | KOH | 99.8 |
| 2 | LiOH•H₂O | 99 |
| 3 | K₂CO₃ | >99.9 |
| 4 | Na₂CO₃ | >99.9 |
| 5 | K₃PO₄ | >99.9 |
| 6 | tBuOK | 97 |

### Example 3: Preparing Propofol by Decarboxylation in Water in Batches

4-hydroxy-3,5diisopropylbenzoic acid (35.6 g, 160.2 mmol, 1 equiv) is suspended in water (70 mL) and stirred at room temperature. An aqueous NaOH solution at 20% weight/volume (100 mL, 320.3 mmol, 2 equiv) is slowly poured in while maintaining the temperature at 20° C. The reaction medium is held with stirring for 30 min and then introduced into a 250 mL autoclave. The reaction medium is heated at 140° C. for 8 h. The reaction mixture is cooled to 60° C., then transferred into a dropping funnel and the organic phase is then separated from the aqueous phase. Crude propofol is obtained with a yield of 85% and an HPLC purity of 97.2%.

### Example 4: Preparing Propofol by Decarboxylation in Water in Continuous Flow

4-hydroxy-3,5-diisopropylbenzoic acid (10 g, 45 mmol, 1 equiv) is suspended in water (22 mL) and stirred at room temperature. An aqueous NaOH solution at 20% weight/volume (20%, 28 mL) is slowly poured in while maintaining the temperature at 20° C. The reaction medium is held with stirring for 30 min and then filtered. The solution obtained is then injected into a continuous flow chemistry system comprising a tubular reactor made of “Hastelloy® C276” alloy with an internal diameter of 1.5 mm and a volume of 5 mL equipped with a double jacket regulated in temperature by a cryothermostat (Huber Ministat 230). The Hastelloy® C276 alloy reactor is pre-heated to the desired temperature and the internal pressure is adjusted to 18 bar using a pressure regulator. The reaction medium is injected using a piston metering pump (Eldex Optos) at pressure P (see Table 3). The residence time in the reactor (tres—see Table 3) is adjusted by means of the pump flow rate. Samples are taken at the outlet of the system after tsampling (=at least 3×tres—see Table 3) and analysed by HPLC (conversion and % propofol: % PFL—see Table 3).

TABLE 3

| T | tres | Flow rate | Tsampling | Pressure | Conversion | |
|---|---|---|---|---|---|---|
| (° C.) | (min) | (mL/min) | (min) | P (PSI) | (%) | % PFL |
| 180 | 5 | 1 | 18 | 264 | 34% | 30.48% |
| 180 | 10 | 0.5 | 30 | 268 | 45% | 39.40% |
| 190 | 5 | 1 | 15 | 264 | 47% | 40.23% |
| 190 | 10 | 0.5 | 30 | 271 | 93% | 90.34% |
| 200 | 5 | 1 | 15 | 274 | 92% | 88.62% |
| 200 | 10 | 0.5 | 30 | 274 | 98% | 93.73% |

1. A process for preparing 2,6-diisopropyl phenol comprising a step of decarboxylation of 4-hydroxy-3,5-diisopropylbenzoic acid or a salt thereof in an aqueous medium under pressure.

2. The process according to claim 1, wherein the decarboxylation step is carried out in the presence of a base.

3. The process according to claim 2, wherein the base is present in a quantity ranging from 0.1 to 3 molar equivalents relative to the quantity of 4-hydroxy-3,5-diisopropylbenzoic acid or a salt thereof.

4. The process according to claim 1, wherein the decarboxylation step is carried out in batches.

5. The process according to claim 1, wherein the decarboxylation step is carried out in batches at a pressure varying from 0.2 MPa to 1.2 MPa.

6. The process according to claim 1, wherein the decarboxylation step is carried out in batches at a temperature ranging from 120° C. to 200° C.

7. The process according to claim 1, wherein the decarboxylation step is carried out in continuous flow.

8. The process according to claim 1, wherein the decarboxylation step is carried out in continuous flow at a pressure ranging from 1.0 MPa to 2.5 MPa.

9. The process according to claim 1, wherein the decarboxylation step is carried out in continuous flow at a temperature ranging from 160° C. to 220° C.

10. The process according to claim 1, wherein the decarboxylation step is carried out in the presence of a mineral base.

11. The process according to claim 1, wherein the decarboxylation step is carried out in the presence of an alkali metal hydroxide.

12. The process according to claim 1, wherein the decarboxylation step is carried out in the presence of a base, the base being present in a quantity ranging from 0.1 to 3 molar equivalents relative to the quantity of 4-hydroxy-3,5-diisopropylbenzoic acid or a salt thereof.

13. The process according to claim 1, wherein the decarboxylation step is carried out in the presence of a base, the base being present in a quantity ranging from 0.5 to 2.5 equivalents relative to the quantity of 4-hydroxy-3,5-diisopropylbenzoic acid or a salt thereof.

14. The process according to claim 1, wherein the decarboxylation step is carried out in batches at a pressure varying from 0.25 MPa to 1.0 MPa.

15. The process according to claim 1, wherein the decarboxylation step is carried in batches at a temperature ranging from 125° C. to 190° C.

16. The process according to claim 1, wherein the decarboxylation step is carried out in continuous flow at a pressure ranging from 1.5 MPa to 2.0 MPa.

17. The process according to claim 1, wherein the decarboxylation step is carried out in continuous flow at a temperature ranging from 170° C. to 210° C.

18. The process according to claim 1, wherein the decarboxylation step is carried out in batches at a pressure varying from 0.2 MPa to 1.2 MPa and at a temperature ranging from 120° C. to 200° C.

19. The process according to claim 1, wherein the decarboxylation step is carried out in continuous flow at a pressure ranging from 1.0 MPa to 2.5 MPa and at a temperature ranging from 160° C. to 220° C.
